(19) United States
(12) Patent Application Publication
(10) Pub. No.: US 2009/0239230 A1
(43) Pub. Date: Sep. 24, 2009

(54) Lung Cancer Diagnosis Based on Expression Levels of GIMAP Genes

(75) Inventors: Shih-Feng Tsai, Miaoll County (TW); Yu-Ming Shiao, Miaoli County (TW); Ya-Hui Chang, Miaoli County (TW); Jih-Shyun Su, Miaoli County (TW)

Correspondence Address: OCCHIUTI ROHLICEK & TSAO, LLP, 10 FAWCETT STREET, CAMBRIDGE, MA, 02138, US

(73) Assignee: National Health Research Institutes, Zhunan Town (TW)

(21) Appl. No.: 12/405,472

(22) Filed: Mar. 17, 2009

Related U.S. Application Data
(60) Provisional application No. 61/037,785, filed on Mar. 19, 2008.

(51) Int. Cl. C12Q 1/68

(52) U.S. Cl. 435/6

(57) ABSTRACT

Diagnosis of lung cancer based on the expression level(s) of one or more GTPase of Immunity-Associated Proteins (GIMAP) and method for identifying anti-lung cancer drug candidates based on their up-regulation of GIMAP activity.

### CROSS-REFERENCE TO RELATED APPLICATION

This application claims the benefit of U.S. Provisional Application 61/037,785, filed Mar. 19, 2008, the content of which is incorporated herein by reference in its entirety.

### BACKGROUND OF THE INVENTION

Lung cancer is the leading cause of cancer death. The efficacy of lung cancer therapy relies heavily on the cancer stage when a treatment is performed. Thus, early diagnosis is key to improving lung cancer therapy. To achieve this mission, one will need to identify molecular markers associated with lung cancer.

The GTPase of immunity-associated proteins (GIMAP), also known as immune-associated nucleotide-binding proteins (IAN), are a conserved GTPase family expressed in vertebrates. See Poirier et al., J. Immunol 163:4960-4969 (1999). They play important roles in regulating immune cell death/survival. See Nitta et al, Trends Immunol 28:58-65 (2006).

### SUMMARY OF THE INVENTION

The present invention is based on the unexpected discovery that the levels of GIMAPs, including GIMAP1, GIMAP2, GIMAP4, GIMAP5, GIMAP6, GIMAP7, and GIMAP8, are significantly lower in human lung tumor tissues than in normal tissues.

Accordingly, one aspect of this invention relates to a method for diagnosing lung cancer. This method includes (1) providing a lung tissue sample obtained from a patient suspected of having lung cancer (e.g., non-small cell lung cancer), (2) examining the expression level of a human GIMAP gene in the lung tissue sample, and (3) determining whether the sample contains tumor material based on the expression level of the gene. A lower expression level of the gene relative to that in a normal lung tissue is indicative that the sample contains tumor material. In one example, the expression level of the GIMAP gene is examined by detecting the level of the mRNAs transcribed from the gene by real-time quantitative polymerase chain reaction (PCR). In another example, the gene expression level is determined by examining the level of the GIMAP by immunohistochemistry staining.

Another aspect of this invention relates to a screening method to identify drug candidates for treating lung cancer. This method includes (1) contacting a compound with lung cancer cells (e.g., those isolated from a lung cancer patient who has not been treated by chemotherapy), (2) detecting in the cancer cells the activity of a human GIMAP, and (3) determining whether the compound is an anti-lung cancer drug candidate based on the GIMAP activity. The compound is a drug candidate for treating lung cancer if it increases the activity of the GIMAP (e.g., the mRNA or protein level of the GIMAP) as compared to that in the lung cancer cells not treated by the compound. In one example, the screening method of this invention is a high throughput assay in which a plurality of compounds are tested.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The drawings are first described.

FIG. 1 is a diagram showing the map of seven human GIMAP genes on chromosomal 7 (upper panel) and their reduced expression levels in lung tumor tissues relative to those in normal tissues (bottom panel). The reduction fold of the expression level of each GIMAP gene was calculated by the formula: [normalized 10g2 value of tumor tissues (T)]/[normalized log₂ value of adjacent non-tumor tissues (N)].

FIGS. 2a-2d are diagrams each showing the mRNA level of GIMAP4, GIMAP5, GIMAP6, or GIMAP8 in lung cancer samples. The mRNA levels of these GIMAPs in both tumor tissues and adjacent normal tissues were determined by the TaqMan Gene Expression Assay. The log₂ ratio refers to the mRNA level of each GIMAP in tumor tissues relative to that in normal tissues.

### DETAILED DESCRIPTION OF THE INVENTION

As pointed out above, we have discovered that the expression levels of all GIMAPs, including GIMAP1, GIMAP2, GIMAP4, GIMAP5, GIMAP6, GIMAP7, and GIMAP8, are significantly reduced in human lung tumor tissues as compared to the nearby non-tumor tissues. Thus, any of the GIMAPs listed above can be used as a molecular marker for diagnosing lung cancer.

Accordingly, disclosed herein is a diagnostic method for determining whether a patient has lung cancer by examining the expression level(s) of one or more GIMAPs in a lung tissue obtained from that patient. Summarized in Table 1 below are GenBank Accession Numbers for both the amino acid sequences and gene sequences of the human GIMAPs:

TABLE 1
GenBank Accession Numbers for Human GIMAPs

| GIMAPs | Protein | Gene |
|---|---|---|
| GIMAP1 | NP_570115 (Jan. 25, 2009) | NM_130759 (Jan. 25, 2009) |
| (IAN2) | | |
| GIMAP2 | NP_056475 (Aug. 7, 2008) | NM_015660 (Aug. 7, 2008) |
| (IAN12) | | |
| GIMAP4 | NP_060796 (Jan. 25, 2009) | NM_018326 (Jan. 25, 2009) |
| (IAN1) | | |
| GIMAP5 | NP_060854 (Mar. 16, 2008) | NM_018384 (Mar. 16, 2008) |
| (IAN5) | | |
| GIMAP6 | NP_078987 (Sep. 14, 2008) | NM_024711 (Sep. 14, 2008) |
| (IAN6) | | |
| GIMAP7 | NP_694968 (Feb. 11, 2008) | NM_153236 (Feb. 11, 2008) |
| (IAN7) | | |
| GIMAP8 | NP_783161 (Feb. 11, 2008) | NM_175571 (Feb. 11, 2008) |
| (IAN9) | | |

To practice the diagnostic method mentioned above, a lung tissue suspected of containing tumor material is obtained from a patient and examined for the expression level of one or more GIMAPs. The expression level of a GIMAP can be determined by examining the protein or mRNA level of the GIMAP by conventional methods, e.g., immunostaining, microarray, or quantitative PCR. Alternatively, the level of a GIMAP can be determined by examining the activity of that GIMAP. If the level of a GIMAP in the lung tissue sample is lower than that in a normal lung tissue (e.g., a normal lung tissue in the same patient), that patient is diagnosed as having lung cancer.

Also disclosed herein is a screening method of determining whether a compound is a drug candidate for treating lung cancer. In this method, a test compound is incubated with human lung cancer cells for a predetermined period of time and the cancer cells are examined for the level of a GIMAP by conventional methods. If the compound up-regulates the GIMAP, i.e., increasing its transcription, stability, or activity, that compound is a candidate drug for treating lung cancer. The lung cancer cells used in this method can be derived from a lung cancer patient who has not been treated by chemotherapy.

The screening method of this invention can be carried out in high throughput format, starting from a combinatorial compound library. The libraries can be spatially addressable parallel solid phase or solution phase libraries. See, e.g., Zuckermann et al. J. Med. Chem. 37, 2678-2685, 1994; and Lam Anticancer Drug Des. 12:145, 1997. Methods for the synthesis of combinatorial compound libraries are well known in the art, e.g., DeWitt et al. PNAS USA 90:6909, 1993; Erb et al. PNAS USA 91:11422, 1994; Zuckermann et al. J. Med. Chem. 37:2678, 1994; Cho et al. Science 261:1303, 1993; Carrell et al. Angew Chem. Int. Ed. Engl. 33:2059, 1994; Carell et al. Angew Chem. Int. Ed. Engl. 33:2061, 1994; and Gallop et al. J. Med. Chem. 37:1233, 1994. Libraries of compounds may be presented in solution (e.g., Houghten Biotechniques 13:412-421, 1992), or on beads (Lam Nature 354:82-84, 1991), chips (Fodor Nature 364:555-556, 1993), bacteria (U.S. Pat. No. 5,223,409), spores (U.S. Pat. No. 5,223,409), plasmids (Cull et al. PNAS USA 89:1865-1869, 1992), orphages (Scott and Smith Science 249:386-390, 1990; Devlin Science 249:404-406, 1990; Cwirla et al. PNAS USA 87:6378-6382, 1990; Felici J. Mol. Biol. 222:301-310, 1991; and U.S. Pat. No. 5,223,409). Upon multiple rounds of screening as described above, compounds that up-regulate GIMAP activity can be identified.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference.

### EXAMPLE 1

### Determination of GIMAP Levels in Non-Small Cell Lung Cancer With Microarray

Tumorous tissues and the adjacent normal tissues were obtained from six patients suffering from non-small cell lung cancer. Among them, one patient had squamous cell carcinoma and the rest five patients had adenocarcinoma. Three of the six patients had an L858R mutation in the EGFR gene.

Total RNAs were isolated from these tissues using TRI REAGENT (Sigma-Aldrich, USA) following the manufacturer's instructions. RNA sample N_Lung A, obtained from Ambion (Ambion Inc., USA), RNA samples N_Lung_B 1 and N_Lung_B2, obtained from BioChain (BioChain Institute, Inc., USA), were used as controls. The quality of these RNAs were examined by the Agilent Bioanalyzer 2100 (Agilent, Palo Alto, Calif.) and those having RNA Integrity Scores larger than five were subjected to the microarray analysis described below.

The HumanRef-8 v2 Expression BeadChip (Illumina, San Diego, Calif.) was used in this study following the manufacturer's instructions to identify genes that showed differential expression patterns in tumor tissues versus in the adjacent normal tissues. Briefly, 500 ng total RNAs were amplified by T7 RNA polymerase using the TotalPrep™ RNA Labeling Kit obtained from Ambion (Ambion Inc., USA). The resultant biotin-labeled cRNAs were applied onto the HumanRef-8 v2 Expression BeadChip, which contains to at least 22,000 transcript probes of human reference sequences (NCBI RefSeq, Release 17) and also negative controls. After hybridization, washing, blocking, and streptavadin-Cy3 staining, the chip was examined for the fluorescence signals released therefrom, which were imaged using the Illumina's BeadArray Reader (See Kuhn K et al., Genome Res 14:2347-2356, 2004). The levels of the signals thus detected (detection p value) were relied on to determine the expression level of each of the genes using BeadStudio software 2.3.4 (Illumina, San Diego, Calif.). More specifically, they were computed based on the Z-values of the genes relative to the Z-values of the negative controls. The gene expression data were normalized (per chip and per gene) by the GeneSpring software 7.3.1 (Agilent, Palo Alto, Calif.).

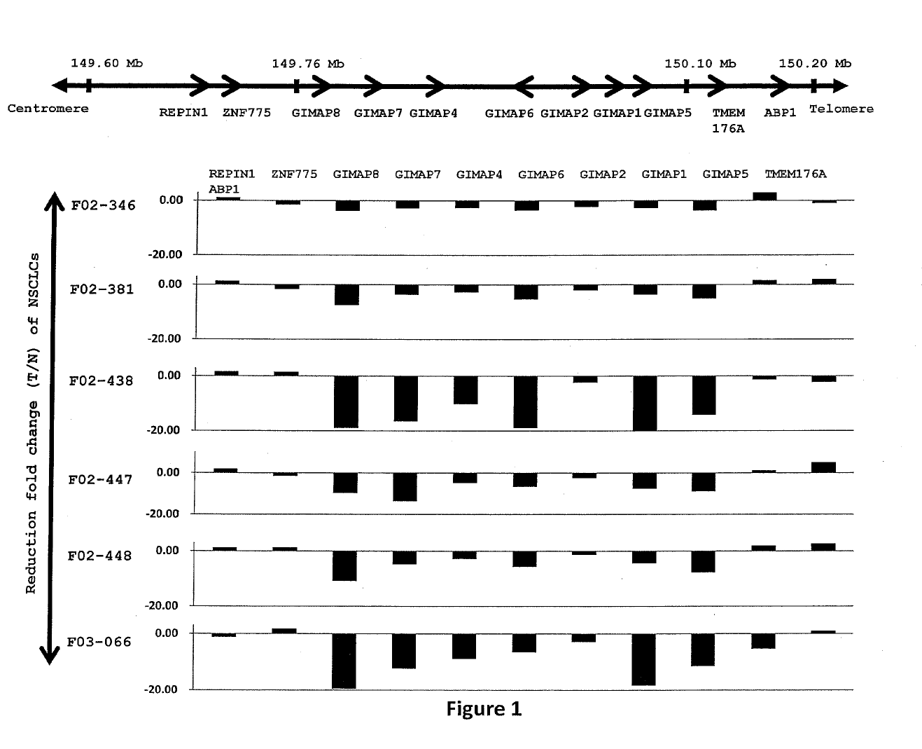

All of the seven members of the GIMAP family, GIMAP1, GIMAP2, GIMAP4, GIMAP5, GIMAP6, GIMAP7, and GIMAP8, showed reduced expression levels in the tumor tissues as compared to their adjacent normal tissues. See FIG. 1. By contrast, this differentiated expression pattern was not observed in genes flanking the GIMAP locus (see FIG. 1).

### EXAMPLE 2

### Determination of GIMAP Levels in Non-Small Cell Lung Cancer with Quantitative Polymerase Chain Reaction

Tumorous tissues and the adjacent normal tissues were obtained from five of the six patients mentioned above and from additional fifteen patients suffering from non-small cell lung cancer. RNAs were extracted from these tissue samples using TRI REAGENT (Sigma-Aldrich, USA) following the manufacturer's instructions. The three RNA samples, i.e., N_Lung_A, N_Lung_B1, and N_Lung_B2 were used as controls.

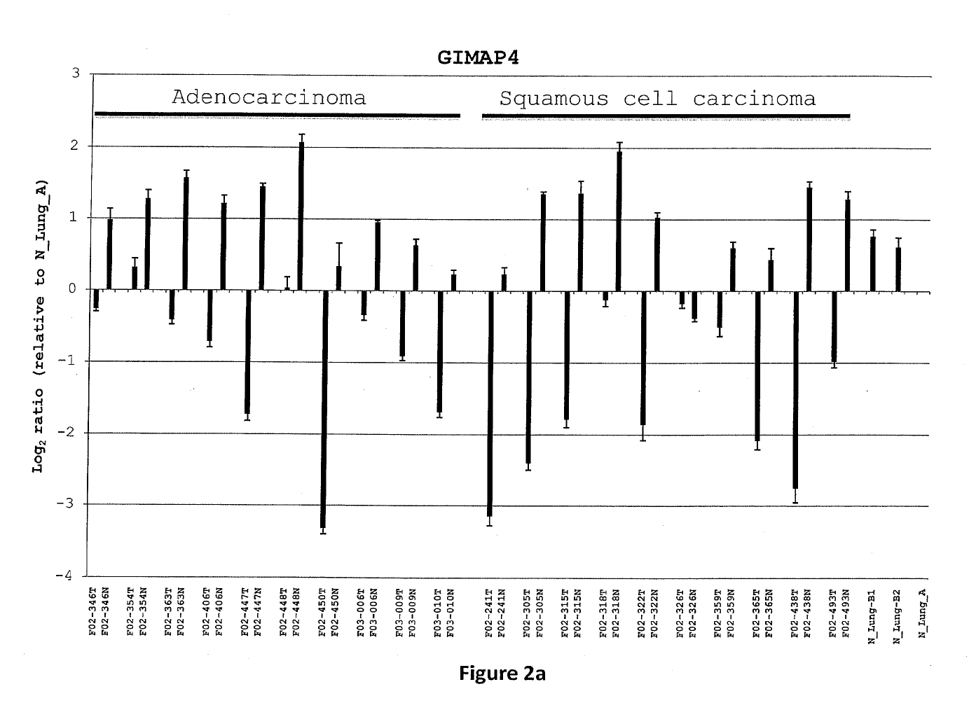

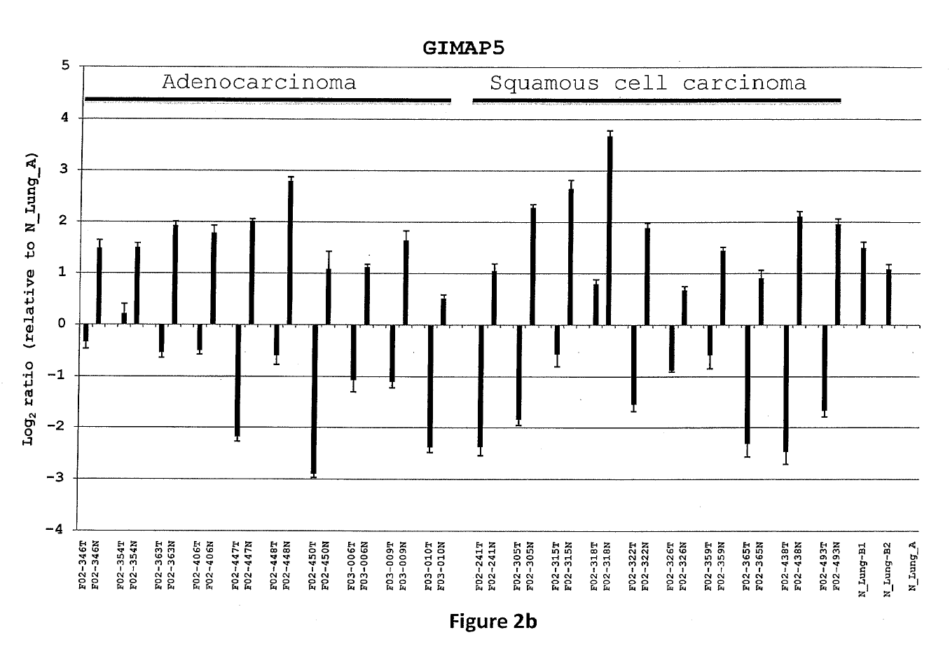

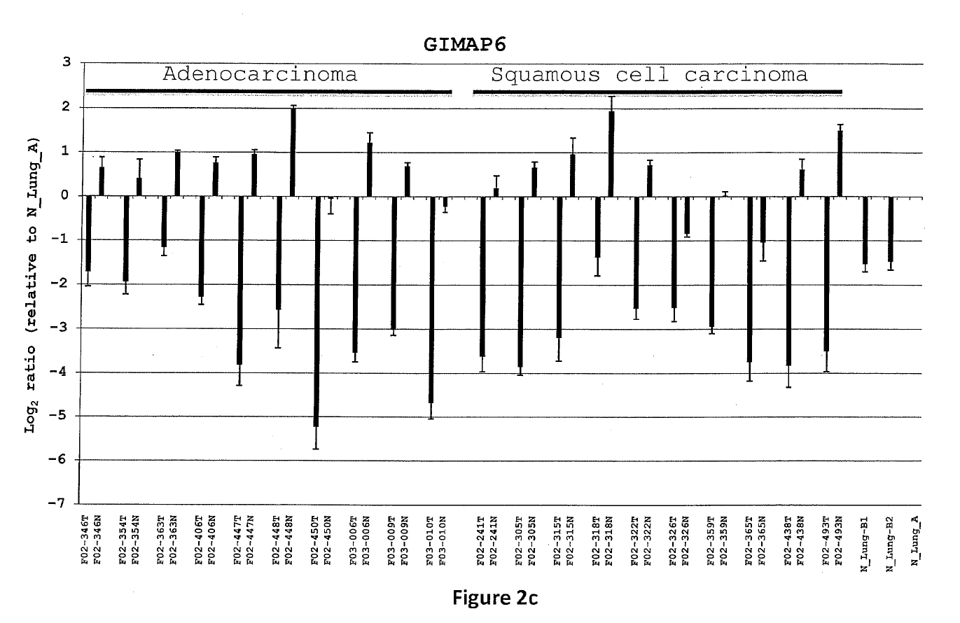

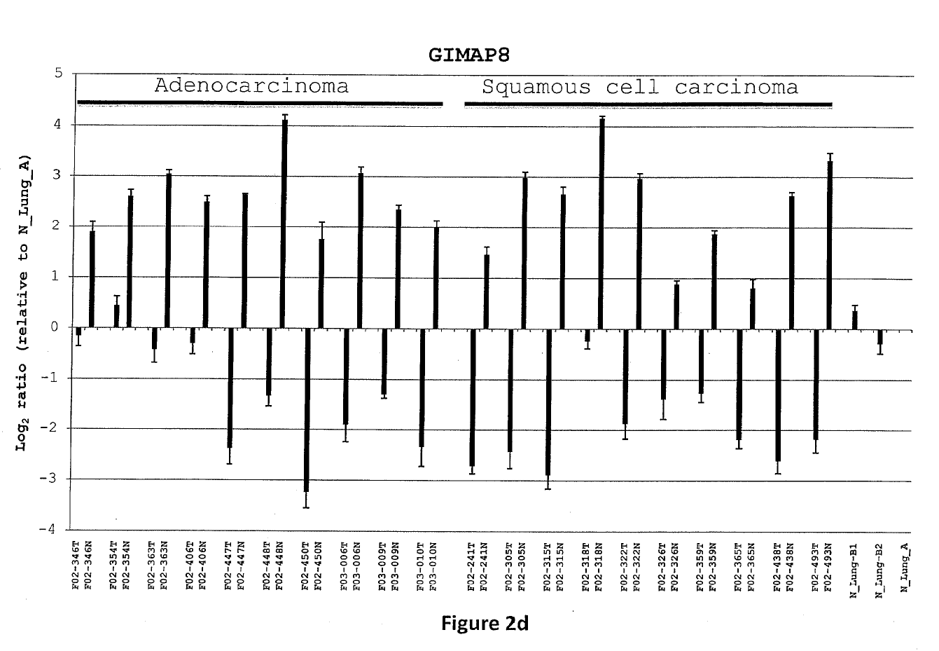

These RNAs were subjected to reverse-transcribed PCR with Transcriptor Reverse Transcriptase (Roche Applied Science, Germany). The cDNAs thus obtained were then analyzed by quantitative PCR to determine the expression levels of GIMAP1, GIMAP2, GIMAP4, GIMAP5, GIMAP6, GIMAP7, GIMAP8, and ACTB (beta-actin, as a control), using TaqMan Gene Expression assay (Applied Biosystems, Foster City, Calif.). The fluorescent signals of each of the RNA samples, representing the expression levels the genes, were measured by the ABI 7900HT system (Applied Biosystems, Foster City, Calif.). The gene expression level of each GIMAP was normalized against that of ACTB. As shown in Table 2 below and in FIGS. 2a-2d, the expression levels of GIMAP1, GIMAP2, GIMAP4, GIMAP5, GIMAP6, GIMAP7, and GIMAP8 in tumor tissues were significantly reduced as compared to their expression levels in the adjacent normal tissues.

TABLE 2
Expression levels of GIMAPs gene in NSCLCs relative to control lung tissues

| | GIMAP1 | GIMAP2 | GIMAP4 | GIMAP5 | GIMAP6 | GIMAP7 | GIMAP8 |
|---|---|---|---|---|---|---|---|
| Nontumor | 1.72 | 2.79 | 2.64 | 2.59 | 4.75 | 3.81 | 11.44 |
| tissue/control | | | | | | | |
| tissue¹ | | | | | | | |
| Tumor | 0.45 | 1.01 | 0.67 | 0.41 | 0.41 | 0.57 | 1.04 |
| tissue/control | | | | | | | |
| tissue² | | | | | | | |
| Nontumor | 7.06 | 5.32 | 6.84 | 10.35 | 18.08 | 11.35 | 19.89 |
| tissue/tumor | | | | | | | |
| tissue³ | | | | | | | |
| Significance | 2.12 × 10⁻⁷ | 1.36 × 10⁻⁴ | 5.02 × 10⁻⁷ | 7.37 × 10⁻⁹ | 6.53 × 10⁻¹⁰ | 3.29 × 10⁻⁷ | 1.33 × 10⁻⁸ |
| (p value)⁴ | | | | | | | |

¹Ratios between the mean gene expression levels in the non-tumor tissues and the mean gene expression levels in the control samples.
²Ratios between the mean gene expression levels in the tumor tissues and the mean gene expression levels in the control samples.
³Ratios between the mean gene expression levels in the non-tumor tissues and the mean gene expression levels in the tumor tissues.
⁴p values were calculated by the Mann-Whitney U test by comparing the relative fold changes of the gene expression levels in the non-tumor tissues versus those in the tumor tissues.

The expression levels of GIMAP1, GIMAP2, GIMAP4, GIMAP5, GIMAP6, GIMAP7, and GIMAP8 in the following seven adenocarcinoma lung cell lines were also determined by quantitative PCR following the method described above: VL80, PE-089, VL107, NCI-H322, NCI-H358, NCI-H1650, and NCI-H1975. The first three cell lines are primary cancer cell lines derived from patients having lung adenocarcinoma and the remaining four are established cancer cell lines. These lung cancer cell lines were cultured in Dulbecco's Modified Eagle's Medium (Invitrogen-Gibco, USA) supplemented with 10% fetal bovine serum (Invitrogen-Gibco, USA). human acute monocytic leukemia cell line (THP-1, BCRC 60430) was used as a control cell line. This cell line was cultured in RPMI 1640 medium (Invitrogen-Gibco, USA) supplemented with 10% fetal bovine serum. The data obtained from these cancer cell lines were compared with that obtained from the THP-1 cell line.

The expression of GIMAP1, GIMAP4, GIMAP5, GIMAP6, GIMAP7, or GIMAP8 was not detected in all of the lung cancer cell lines and GIMAP2 was expressed at an extremely low level. Differently, expression of GIMAP5, GIMAP6, and GIMAP8 was detected in the THP-1 cell line.

### EXAMPLE 3

### Determination of GIMAP4 Expression in Tumor Tissues and Non-Tumor Tissues with Immunohistochemistry Staining

The tumor and non-tumor tissues, obtained from lung cancer patients (having squamous cell carcinoma or adenocarcinoma), were fixed with 10% formalin, embedded in paraffin, and then cut into 5-μm sections for immunohistochemical staining. The sections were de-waxed, re-hydrated stepwise with graded ethanol solutions, and washed with double-distilled water. Before staining, the sections were treated with the Microwave Vacuum Histoprocessor RHS-1 (Milestone Inc., Shelton, Conn., USA) for antigen retrieval and also treated with 3% hydrogen-peroxide-methanol solution to quench endogenous peroxidase activity. The sections were then incubated at 4° C. overnight with a murine anti-human GIMAP4 monoclonal antibody (ProteinTech Group, Inc, Chicago, USA), diluted at 1:100 in 0. 15 M phosphate buffered saline (PBS). They were then inducated with a biotinylated anti-murine IgG antibody (DAKO Cytomation LSAB2 System-HRP; DAKO, Copenhagen, Denmark) and subsequently with a streptavidin-alkaline phosphatase (ALP) IgG conjugate. After being washed for several times, the sections were incubated with the DAB substrate for color development, using the DAB Substrate Kit (DAKO Cytomation Liquid DAB+ substrate chromogen system; DAKO, Copenhagen, Denmark).

Positive immunostaining signals were detected in the normal tissues (in lymphocytes, endothelial cells, and histiocytes), but not in the tumor tissues.

### Other Embodiments

All of the features disclosed in this specification may be combined in any combination.

Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method of diagnosing lung cancer, comprising
providing a lung tissue sample obtained from a patient suspected of having lung cancer,
examining the expression level of a gene encoding a human GTPase of immunity-associated protein (GIMAP) in the lung tissue sample, and
determining whether the lung tissue sample contains tumor material based on the expression level of the gene,
wherein the expression level of the gene lower than that in a normal lung tissue indicates that the lung tissue sample contains tumor material.

2. The method of claim 1, wherein the GIMAP is human GIMAP1, GIMAP2, GIMAP4, GIMAP5, GIMAP6, GIMAP7, or GIMAP8.

3. The method of claim 2, wherein the GIMAP is human GIMAP6.

4. The method of claim 2, wherein the GIMAP is human GIMAP8.

5. The method of claim 1, wherein the patient is suspected of having non-small cell lung cancer.

6. The method of claim 1, wherein the expression level of the gene is examined by detecting the level of the mRNA transcribed from the gene by real-time quantitative polymerase chain reaction.

7. The method of claim 1, wherein the expression level of the gene is examined by detecting the level of the protein encoded by the gene by immunohistochemistry staining.

8. The method of claim 3, wherein the patient is suspected of having non-small cell lung cancer.

9. The method of claim 3, wherein the expression level of GIMAP6 is examined by detecting the mRNA level of GIMAP6 by real-time quantitative polymerase chain reaction.

10. The method of claim 3, wherein the expression level of GIMAP6 is examined by detecting the protein level of GIMAP6 by immunohistochemistry staining.

11. The method of claim 4, wherein the patient is suspected of having non-small cell lung cancer.

12. The method of claim 4, wherein the expression level of GIMAP8 is examined by detecting the mRNA level of GIMAP8 by real-time quantitative polymerase chain reaction.

13. The method of claim 4, wherein the expression level of GIMAP8 is examined by detecting the protein level of GIMAP8 by immunohistochemistry staining.

14. A method for determining whether a compound is a drug candidate for treating lung cancer, comprising
contacting a compound with lung cancer cells,
detecting in the cancer cells the activity of a human GTPase of immunity-associated protein (GIMAP), and
determining whether the compound is an anti-lung cancer drug candidate based on the level of the GIMAP activity,
wherein an increased activity of the GIMAP, as compared to that in the lung cancer cells not treated by the compound, indicates that the compound is an anti-lung cancer drug candidate.

15. The method of claim 14, wherein the lung cancer cells is isolated from a lung cancer patient who has not been treated by chemotherapy.

16. The method of claim 14, wherein the GIMAP is human GIMAP1, GIMAP2, GIMAP4, GIMAP5, GIMAP6, GIMAP7, or GIMAP8.

17. The method of claim 16, wherein the GIMAP is human GIMAP6 or GIMAP8.

18. The method of claim 14, wherein the activity of the GIMAP is detected by examining the mRNA level of the GIMAP by real-time quantitative polymerase chain reaction.

19. The method of claim 14, wherein the activity of the GIMAP is detected by examining the protein level of the GIMAP by immunohistochemistry staining.

20. The method of claim 14, wherein the lung cancer cells are non-small cell lung cancer cells.

21. The method of claim 14, wherein the lung cancer cells are small cell lung cancer cells.

22. The method of claim 14, wherein the method is a high throughput screening assay, in which a plurality of compounds are tested.
